(19) United States
(12) Patent Application Publication
(10) Pub. No.: US 2025/0281425 A1
(43) Pub. Date: Sep. 11, 2025

(54) METHODS FOR TREATING SKIN DISORDERS WITH A TOPICAL COMPOSITION OF BIS-(2-CHLOROETHYL)METHYLAMINE

(71) Applicant: HELSINN BIREX PHARMACEUTICALS LIMITED, Dublin (IE)

(72) Inventors: Etienne KRAUSBAUER, Allschwil (CH); Olivier LAMBERT, Allschwil (CH); Frederic NAUD, Allschwil (CH); Dalia RAYES, South San Francisco, CA (US); Celine SALAVERT, Allschwil (CH)

(73) Assignee: HELSINN BIREX PHARMACEUTICALS LIMITED, Dublin (IE)

(21) Appl. No.: 19/218,513

(22) Filed: May 26, 2025

Related U.S. Application Data
(63) Continuation of application No. 15/804,804, filed on Nov. 6, 2017, now abandoned.
(60) Provisional application No. 62/418,606, filed on Nov. 7, 2016.

(51) Int. Cl. A61K 31/131; A61K 9/00; A61K 9/06; A61K 47/02; A61K 47/10; A61K 47/12; A61K 47/18; A61K 47/38

(52) U.S. Cl. CPC A61K 31/131; A61K 9/0014; A61K 9/06; A61K 47/02; A61K 47/10; A61K 47/12; A61K 47/183; A61K 47/38

(57) ABSTRACT

Provided are methods for treating skin disorders comprising topically applying a highly stable pharmaceutical composition comprising bis-(2-chloroethyl)methylamine or a pharmaceutically acceptable salt thereof.

### CROSS REFERENCE TO RELATED APPLICATIONS

This application claims priority to and the benefit of U.S. Provisional Application No. 62/418,606, filed Nov. 7, 2016, the contents of each of which are incorporated herein by reference.

### FIELD OF THE INVENTION

The invention encompasses methods for treating skin disorders comprising topically applying a highly stable pharmaceutical composition comprising bis-(2-chloroethyl)methylamine or a pharmaceutically acceptable salt thereof.

### BACKGROUND OF THE INVENTION

Alkylating agents, such as nitrogen mustards (especially bis-(2-chloroethyl)methylamine), have been used in the pharmaceutical industry as anti-cancer drugs. For example, nitrogen mustards have been used to treat cutaneous T-cell lymphoma (CTCL), including mycosis fungoides (MF).

CTCL is a cancer of the white blood cells that primarily affects the skin and only secondarily affects other sites. The disease involves the uncontrolled proliferation of T-lymphocytes known as T-helper (CD4+) cells of the immune system. The proliferation of T-helper cells results in the penetration, or infiltration, of these abnormal cells into the epidermal layer of the skin. The skin reacts with slightly scaling lesions that itch, although the sites of greatest infiltration do not necessarily correspond to the sites of the lesions. The lesions are most often located on the trunk, but can be present on any part of the body. In the most common course of the disease, the patchy lesions progress to palpable plaques that are deeper red and have more defined edges. As the disease worsens, skin tumors develop that are often mushroom-shaped, hence the name mycosis fungoides. Finally, the cancer progresses to extracutanous involvement, often in the lymph nodes or the viscera.

Alkylating agents, such as nitrogen mustards, are believed to have anti-cancer activity by acting on the nucleic acids of DNA and RNA. Alkylating agents have four main actions on nucleic acids. First, the agents may cause crosslinking of DNA strands which interferes with DNA and RNA synthesis. This is thought to be the most important reason for the cytotoxic effect of alkylating agents. Secondly, the agents may alter the “side chain groups” of the nucleotide base ring which would lead to abnormal base pairing and point mutations in the synthesized DNA and RNA chains. Thirdly, the alkylating agents may split the base ring from the nucleotide which again interrupts proper DNA and RNA synthesis. Finally, the alkylating agents may break the ring structure of a nucleotide base which would prevent base pairing during DNA and RNA synthesis.

Nitrogen mustards are believed to act as anti-cancer agents by impairing natural DNA strand replication of cancer cells. In natural DNA strand replication, a DNA strand having deoxyribonucleosides, wherein each deoxyribonucleoside may include a base adenine (A), thymine (T), cytosine (C) and guanine (G), replicates by linking each deoxyribonucleoside on the strand with another deoxyribonucleoside, wherein typical linking occurs between adenine (A) and thymine (T), forming an A-T linkage, and between cytosine (C) and guanine (G), forming a C-G linkage, between the original DNA strand and its replicated DNA strand. Nitrogen mustards are believed to allow unnatural base-base linkages such as a guanine (G) base linking to another guanine (G) base if the particular nitrogen mustard is a bifunctional alkylator. As used herein, unless otherwise defined, a bifunctional alkylator is a nitrogen mustard that has at least two 2-chloroethyl side chains, for example, bis-(2-chloroethyl)methylamine.

Bis-(2-chloroethyl)methylamine is known to be a highly reactive compound that is unstable in various media, such as especially in water (Reepmeyer J C J. Chromatography A (2005), 1085, 262-269; Ritschel W A et al. Int. J. Pharmaceutics (2008), 362, 67-73). For many years topical formulations of bis-(2-chloroethyl)methylamine hydrochloride were thus freshly compounded by a pharmacist either as an aqueous solution or as an ointment, as for instance in petrolatum-based ointments that are greasy, sticky and unpleasant for the patient.

A greaseless pharmaceutical composition for the topical application of bis-(2-chloroethyl)methylamine hydrochloride with an improved stability is described for instance in Sahu et al. Clin. Invest. (2014), 4 (8), 745-761, WO 2006/099385 and WO 2009/120493. The composition has been approved by the U.S. Food and Drug Administration as VALCHLOR® for the topical treatment of Stage IA and IB mycosis fungoides-type cutaneous T-cell lymphoma in patients who have received prior skin-directed therapy. According to the prescribing information the product should be stored by the patient at temperatures between 2° and 8° C. and should not be left at room temperature for longer than one hour per day. Unused product should be discarded after 60 days.

Despite the high reactivity and the limited stability of bis-(2-chloroethyl)methylamine and its hydrochloride salt it has been surprisingly found that a pharmaceutical composition comprising bis-(2-chloroethyl)methylamine hydrochloride and 2-(2-ethoxy-ethoxy)-ethanol is sufficiently stable to be used for at least 90 days when stored at about room temperature for up to 1 hour per day and refrigerated for the remaining time of the day. It is even more surprising that the pharmaceutical composition may be partially exposed to humidity and light during the room temperature period of the aforementioned storage conditions and is still sufficiently stable to be used for at least 90 days; this is of high importance since it reflects the typical conditions the product is used by a patient: storing of a container (such as an aluminum foil tube) containing the composition in a fridge, taking the tube out of the fridge, opening the tube, dispensing and applying the composition, closing the tube and putting the tube back into the fridge. It is beyond doubt that such in-use conditions have a higher impact on the stability of bis-(2-chloroethyl)methylamine and its pharmaceutically acceptable salts than storage conditions wherein a closed container is stored without warming/cooling cycles.

### DETAILED DESCRIPTION OF THE INVENTION

In a first embodiment, the invention relates to a method of treating a skin disorder selected from cutaneous T-cell lymphoma (notably mycosis fungoides), psoriasis, alopecia (notably alopecia areata), lymphomatoid papulosis, parapsoriasis (notably large plaque parapsoriasis), Langerhans cell histiocytosis and vitiligo comprising topically applying a pharmaceutical composition to a subject in need thereof, wherein the pharmaceutical composition comprises an effective amount of bis-(2-chloroethyl)methylamine, or a pharmaceutically acceptable salt thereof, and an excipient that is a compound of the formula HOCH₂CH₂OCH₂CH₂OR, wherein R represents (C₁-C₄)alkyl; and wherein the pharmaceutical composition is essentially stable for at least 90 days and up to 240 days when stored for up to 1 hour per day at about room temperature and for the remaining time of the day at or below 8° C.

In one embodiment, the invention relates to a method of treating mycosis fungoides.

In another embodiment, the invention relates to a method of treating Stage IA and IB mycosis fungoides-type cutaneous T-cell lymphoma.

In a preferred embodiment, the invention relates to a method of treating Stage IA and IB mycosis fungoides-type cutaneous T-cell lymphoma in a subject (notably a patient) who has received prior skin-directed therapy.

In one embodiment, the bis-(2-chloroethyl)methylamine, or the pharmaceutically acceptable salt thereof, is bis-(2-chloroethyl)methylamine hydrochloride.

In one embodiment, the bis-(2-chloroethyl)methylamine, or the pharmaceutically acceptable salt thereof, is present in an amount of about 0.005% to about 0.1% by weight of the composition.

In another embodiment, the bis-(2-chloroethyl)methylamine, or the pharmaceutically acceptable salt thereof, is present in an amount of about 0.01% to about 0.05% by weight of the composition.

In still another embodiment, the bis-(2-chloroethyl)methylamine, or the pharmaceutically acceptable salt thereof, is present in an amount of about 0.02% by weight of the composition.

In one embodiment, the pharmaceutical composition does not contain petrolatum.

In one embodiment, the pharmaceutical composition comprises less than 2% by weight water; preferably less than 1% by weight water.

In one embodiment, the pharmaceutical composition comprises less than 3% by weight ethanol; preferably less than 1% by weight ethanol. Most preferably, the pharmaceutical composition does not contain ethanol.

In one embodiment, the compound of the formula HOCH₂CH₂OCH₂CH₂OR is the compound HOCH₂CH₂OCH₂CH₂OCH₂CH₃ (2-(2-ethoxy-ethoxy)-ethanol).

In one embodiment, the compound of the formula HOCH₂CH₂OCH₂CH₂OR (and notably the compound of the formula HOCH₂CH₂OCH₂CH₂OCH₂CH₃) is present in an amount of about 40% to about 60% by weight of the composition.

In one embodiment, the pharmaceutical composition is essentially stable for at least 90 days and up to 240 days when stored for up to 1 hour per day at about room temperature and for the remaining time of the day between −25° and 8° C.

In another embodiment, the pharmaceutical composition is essentially stable for at least 90 days and up to 240 days when stored for up to 1 hour per day at about room temperature and for the remaining time of the day between 2° and 8° C.

In one embodiment, the pharmaceutical composition is essentially stable for at least 90 days and up to 180 days when stored for up to 1 hour per day at about room temperature and for the remaining time of the day at or below 8° C.

In another embodiment, the pharmaceutical composition is essentially stable for at least 90 days and up to 180 days when stored for up to 1 hour per day at about room temperature and for the remaining time of the day between 2° and 8° C.

In one embodiment, the pharmaceutical composition is essentially stable for at least 90 days and up to 168 days when stored for up to 1 hour per day at about room temperature and for the remaining time of the day at or below 8° C.

In another embodiment, the pharmaceutical composition is essentially stable for at least 90 days and up to 168 days when stored for up to 1 hour per day at about room temperature and for the remaining time of the day between 2° and 8° C.

In one embodiment, the pharmaceutical composition is essentially stable for at least 90 days and up to 112 days when stored for up to 1 hour per day at about room temperature and for the remaining time of the day at or below 8° C.

In another embodiment, the pharmaceutical composition is essentially stable for at least 90 days and up to 112 days when stored for up to 1 hour per day at about room temperature and for the remaining time of the day between 2° and 8° C.

In one embodiment, the pharmaceutical composition is essentially stable for 90 days when stored for up to 1 hour per day at about room temperature and for the remaining time of the day at or below 8° C.

In another embodiment, the pharmaceutical composition is essentially stable for 90 days when stored for up to 1 hour per day at about room temperature and for the remaining time of the day between 2° and 8° C.

In one embodiment, at least 90% of the original amount of the bis-(2-chloroethyl)methylamine, or of the pharmaceutically acceptable salt thereof, is still present in the pharmaceutical composition, if the pharmaceutical composition is stored at about room temperature for up to 1 hour per day and at 8° C. or below for the remaining time of the day for a total of 90 days.

In another embodiment, at least 90% of the original amount of the bis-(2-chloroethyl)methylamine, or of the pharmaceutically acceptable salt thereof, is still present in the pharmaceutical composition, if the pharmaceutical composition is stored at about room temperature for up to 1 hour per day and at between 2° and 8° C. for the remaining time of the day for a total of 90 days.

In one embodiment, at least 90% of the original amount of the bis-(2-chloroethyl)methylamine, or of the pharmaceutically acceptable salt thereof, is still present in the pharmaceutical composition, if the pharmaceutical composition is stored first at a temperature between −25° to −15° C. for up to 6 month; and second at about room temperature for up to 1 hour per day and at 8° C. or below (notably between 2° and 8° C.) for the remaining time of the day for a total of 90 days.

In another embodiment, at least 90% of the original amount of the bis-(2-chloroethyl)methylamine, or of the pharmaceutically acceptable salt thereof, is still present in the pharmaceutical composition, if the pharmaceutical composition is stored first at a temperature between −25° to −15° C. for up to 18 month; and second at about room temperature for up to 1 hour per day and at 8° C. or below (notably between 2° and 8° C.) for the remaining time of the day for a total of 90 days.

In still another embodiment, at least 90% of the original amount of the bis-(2-chloroethyl)methylamine, or of the pharmaceutically acceptable salt thereof, is still present in the pharmaceutical composition, if the pharmaceutical composition is stored first at a temperature between −25° to −15°° C. for up to 36 month; and second at about room temperature for up to 1 hour per day and at 8° C. or below (notably between 2° and 8° C.) for the remaining time of the day for a total of 90 days.

In one embodiment according to any one of the three aforementioned embodiments, the pharmaceutical composition is protected from humidity and light during the first storage at a temperature between −25° to −15° C. and is partially exposed to humidity and/or light during the period the pharmaceutical composition is stored at about room temperature.

In one embodiment, the pharmaceutical composition comprises bis-(2-chloroethyl)methylamine, or a pharmaceutically acceptable salt thereof (notably bis-(2-chloroethyl)methylamine hydrochloride), and a compound of the formula HOCH₂CH₂OCH₂CH₂OR, wherein R represents (C₁-C₄)alkyl (notably 2-(2-ethoxy-ethoxy)-ethanol), and further comprises hydroxypropyl cellulose.

In one embodiment, the pharmaceutical composition comprises bis-(2-chloroethyl)methylamine, or a pharmaceutically acceptable salt thereof (notably bis-(2-chloroethyl)methylamine hydrochloride), and a compound of the formula HOCH₂CH₂OCH₂CH₂OR, wherein R represents (C₁-C₄)alkyl (notably 2-(2-ethoxy-ethoxy)-ethanol), and further comprises butylated hydroxytoluene.

In one embodiment, the pharmaceutical composition comprises bis-(2-chloroethyl)methylamine, or a pharmaceutically acceptable salt thereof (notably bis-(2-chloroethyl)methylamine hydrochloride), and a compound of the formula HOCH₂CH₂OCH₂CH₂OR, wherein R represents (C₁-C₄)alkyl (notably 2-(2-ethoxy-ethoxy)-ethanol), and further comprises isopropyl alcohol.

In one embodiment, the pharmaceutical composition comprises bis-(2-chloroethyl)methylamine, or a pharmaceutically acceptable salt thereof (notably bis-(2-chloroethyl)methylamine hydrochloride), and a compound of the formula HOCH₂CH₂OCH₂CH₂OR, wherein R represents (C₁-C₄)alkyl (notably 2-(2-ethoxy-ethoxy)-ethanol), and further comprises lactic acid.

In one embodiment, the pharmaceutical composition comprises bis-(2-chloroethyl)methylamine, or a pharmaceutically acceptable salt thereof (notably bis-(2-chloroethyl)methylamine hydrochloride), and a compound of the formula HOCH₂CH₂OCH₂CH₂OR, wherein R represents (C₁-C₄)alkyl (notably 2-(2-ethoxy-ethoxy)-ethanol), and further comprises hydroxypropyl cellulose, butylated hydroxytoluene, isopropyl alcohol and lactic acid.

In one embodiment, the pharmaceutical composition comprises bis-(2-chloroethyl)methylamine, or a pharmaceutically acceptable salt thereof (notably bis-(2-chloroethyl)methylamine hydrochloride), and a compound of the formula HOCH₂CH₂OCH₂CH₂OR, wherein R represents (C₁-C₄)alkyl (notably 2-(2-ethoxy-ethoxy)-ethanol), and further comprises hydroxypropyl cellulose, edetate disodium (notably edetate disodium dihydrate), menthol, butylated hydroxytoluene, isopropyl alcohol, propylene glycol, glycerin, lactic acid, and sodium chloride.

In one embodiment, the pharmaceutical composition comprises bis-(2-chloroethyl)methylamine hydrochloride and 2-(2-ethoxy-ethoxy)-ethanol, and further comprises about 1% to about 3% by weight of the composition hydroxypropyl cellulose.

In one embodiment, the pharmaceutical composition comprises bis-(2-chloroethyl)methylamine hydrochloride and 2-(2-ethoxy-ethoxy)-ethanol, and further comprises about 0.005% to about 0.02% by weight of the composition edetate disodium dihydrate.

In one embodiment, the pharmaceutical composition comprises bis-(2-chloroethyl)methylamine hydrochloride and 2-(2-ethoxy-ethoxy)-ethanol, and further comprises about 0.01% to about 0.1% by weight of the composition menthol.

In one embodiment, the pharmaceutical composition comprises bis-(2-chloroethyl)methylamine hydrochloride and 2-(2-ethoxy-ethoxy)-ethanol, and further comprises about 0.005% to about 0.02% by weight of the composition butylated hydroxytoluene.

In one embodiment, the pharmaceutical composition comprises bis-(2-chloroethyl)methylamine hydrochloride and 2-(2-ethoxy-ethoxy)-ethanol, and further comprises about 10% to about 20% by weight of the composition isopropyl alcohol.

In one embodiment, the pharmaceutical composition comprises bis-(2-chloroethyl)methylamine hydrochloride and 2-(2-ethoxy-ethoxy)-ethanol, and further comprises about 13% to about 23% by weight of the composition propylene glycol.

In one embodiment, the pharmaceutical composition comprises bis-(2-chloroethyl)methylamine hydrochloride and 2-(2-ethoxy-ethoxy)-ethanol, and further comprises about 6% to about 16% by weight of the composition glycerin.

In one embodiment, the pharmaceutical composition comprises bis-(2-chloroethyl)methylamine hydrochloride and 2-(2-ethoxy-ethoxy)-ethanol, and further comprises about 2% to about 6% by weight of the composition lactic acid.

In one embodiment, the pharmaceutical composition comprises bis-(2-chloroethyl)methylamine hydrochloride and 2-(2-ethoxy-ethoxy)-ethanol, and further comprises about 0.1% to about 0.3% by weight of the composition sodium chloride.

In one embodiment, the pharmaceutical composition comprises about 0.01% to about 0.05% by weight of the composition bis-(2-chloroethyl)methylamine hydrochloride and about 40% to about 60% by weight of the composition 2-(2-ethoxy-ethoxy)-ethanol, and further comprises:
about 1% to about 3% by weight of the composition hydroxypropyl cellulose;
about 0.005% to about 0.02% by weight of the composition edetate disodium dihydrate;
about 0.01% to about 0.1% by weight of the composition menthol;
about 0.005% to about 0.02% by weight of the composition butylated hydroxytoluene;
about 10% to about 20% by weight of the composition isopropyl alcohol;
about 13% to about 23% by weight of the composition propylene glycol;
about 6% to about 16% by weight of the composition glycerin;
about 2% to about 6% by weight of the composition lactic acid; and
about 0.1% to about 0.3% by weight of the composition sodium chloride.

In one embodiment, the pharmaceutical composition is exposed to humidity and/or light during the period the pharmaceutical composition is stored at about room temperature.

In one embodiment, the pharmaceutical composition is partially exposed to humidity and/or light during the period the pharmaceutical composition is stored at about room temperature.

In one embodiment, the pharmaceutical composition is in the form of a paste, a dispersion, a suspension, a solution, a gel, a cream, or an ointment (notably in form of a gel).

In one embodiment, the pharmaceutical composition is stored in a glass vial. In another embodiment, the composition is stored in an amber vial. In another embodiment, the composition is stored in an aluminum foil-lined container. In another embodiment, the composition is stored in an aluminum foil tube. In another embodiment, the composition is stored in a plastic container. In another embodiment, the composition is stored in a polypropylene container. Preferred is an aluminum foil tube.

In one embodiment, the vial, container or tube, that is used for the storage of the pharmaceutical composition, is a multiple-dose vial, container or tube. With other words the vial, container or tube can be used to dispense more than one dose of the pharmaceutical composition. Notably, the vial, container or tube can be used to dispense the pharmaceutical composition once a day for up to 90 days, for up to 112 days, for up to 168 days, for up to 180 days, or for up to 240 days (especially for up to 90 days).

Definitions provided herein are intended to apply uniformly throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

Where the plural form is used for compounds, salts, pharmaceutical compositions, disorders, diseases and the like, this is intended to mean also a single compound, salt, pharmaceutical composition, disorder, disease or the like.

For the avoidance of doubt, bis-(2-chloroethyl)methylamine is also denoted as mechlorethamine, chlormethine or 2-chloro-N-(2-chloroethyl)-N-methylethan-1-amine, and has the chemical formula:

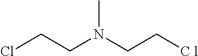

The term “alkyl”, used alone or in combination, refers to a straight or branched chain alkyl group containing one to four carbon atoms. The term “(Cₓ-C_{y})alkyl” (x and y each being an integer), refers to an alkyl group as defined before containing x to y carbon atoms. For example a (C₁-C₄)alkyl group contains from one to four carbon atoms. Examples of said groups are methyl, ethyl, n-propyl, iso-propyl, n-butyl, iso-butyl, sec.-butyl and tert.-butyl; preferred are linear (C₁-C₄)alkyl groups such as methyl, ethyl, n-propyl and n-butyl. Most preferred is ethyl.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-V C H, 2008 and ‘Pharmaceutical Salts and Co-crystals’, Johan Wouters and Luc Quéré (Eds.), RSC Publishing, 2012. Preferred pharmaceutically acceptable salts are acid-addition salts such as HCl, HBr, HNO₃, H₂SO₄, MHSO₄, H₃PO₄, MH₂PO₄ or M₂HPO₄ salts, wherein M represents a pharmaceutically acceptable cation such as an alkali metal cation (especially Na⁺ or K⁺). It is understood that one or more hydrogen cations (up to all hydrogen cations) of acids containing more than one acidic hydrogen atom, such as H₂SO₄, H₃PO₄ or MH₂PO₄, may be replaced by the bis-(2-chloroethyl)methylammonium cation. The most preferred pharmaceutically acceptable salt is the hydrochloride salt.

The present invention also includes isotopically labelled, especially ²H (deuterium) labelled derivatives of bis-(2-chloroethyl)methylamine, which derivatives are identical to bis-(2-chloroethyl)methylamine except that one or more atoms have each been replaced by an atom having the same atomic number but an atomic mass different from the atomic mass usually found in nature. Isotopically labelled, especially ²H (deuterium) labelled derivatives of bis-(2-chloroethyl)methylamine and pharmaceutically acceptable salts thereof are within the scope of the present invention. Substitution of hydrogen with the heavier isotope ²H (deuterium) may lead to greater chemical or metabolic stability, resulting e.g. in increased in-vivo half-life or reduced dosage requirements, or may lead to reduced inhibition of cytochrome P450 enzymes, resulting e.g. in an improved safety profile. In one embodiment of the invention, the bis-(2-chloroethyl)methylamine is not isotopically labelled, or is labelled only with one or more deuterium atoms. In another embodiment, the bis-(2-chloroethyl)methylamine is not isotopically labelled at all (i.e. all isotops are present according to their natural abundance).

Whenever the word “between” is used to describe a numerical range, it is to be understood that the end points of the indicated range are explicitly included in the range. For example: if a temperature range is described to be between 2° and 8° C., this means that the end points 2° C. and 8° C. are included in the range; or if a variable is defined as being an integer between 1 and 4, this means that the variable is the integer 1, 2, 3, or 4.

Unless used regarding temperatures, the term “about” (or alternatively “around”) placed before a numerical value “X” refers in the current application to an interval extending from X minus 10% of X to X plus 10% of X, and preferably to an interval extending from X minus 5% of X to X plus 5% of X. Most preferred is value “X”. In the particular case of temperatures, the term “about” (or alternatively “around”) placed before a temperature “Y” refers in the current application to an interval extending from the temperature Y minus 10° C. to Y plus 10° C., and preferably to an interval extending from Y minus 5° C. to Y plus 5° C. Most preferred is temperature “Y”. Besides, the term “room temperature” as used herein refers to a temperature of 25° C.

If a time specification is described to last for “up to 1 hour”, this means that the endpoint “1 hour” is included; in one embodiment such time specification means a period of time between 10 minutes and 1 hour; in another embodiment such time specification means a period of time between 30 minutes and 1 hour; in still another embodiment such time specification means a period of time between 50 minutes and 1 hour.

In the phrase “when stored for up to 1 hour per day at one temperature and for the remaining time of the day at another temperature” (or in analogous phrases) the term “day” refers to a period of time of 24 hours; with other words the time periods “up to 1 hour per day” and “remaining time of the day” sum to a period of 24 hours.

If a time specification is described to last for “at least 90 days”, this means that the starting point “90 days” is included; with other words, a pharmaceutical composition that is described to be essentially stable for at least 90 days is essentially stable under the given conditions for 90 days or longer.

If a time specification is described to last for “up to 240 days” (or “up to 180 days”, “up to 168 days”, “up to 112 days”, respectively), this means that the end point “240 days” (or “180 days”, “168 days”, “112 days”, respectively) is included.

If a pharmaceutical composition is described to contain “at least 90% of the original amount of the bis-(2-chloroethyl)methylamine”, this means that an amount of 90% or more bis-(2-chloroethyl)methylamine is present in the pharmaceutical composition after storage under the specifically given conditions if compared to the amount as present in a freshly prepared pharmaceutical composition.

The term “topically applying” (or alternatively “topically administering”) means applying a pharmaceutical composition to the surface of the whole body or to the surface of a part of the body of a subject in need thereof. Preferably the pharmaceutical composition is topically applied to the affected areas of the skin.

The term “effective amount” when referring to bis-(2-chloroethyl)methylamine, or a pharmaceutically acceptable salt thereof, means an amount of bis-(2-chloroethyl)methylamine, or of a pharmaceutically acceptable salt thereof, that is effective to treat a skin disorder.

The term “prior skin-directed therapy” especially refers to therapies including topical corticosteroids, phototherapy, Targretin® gel, and topical nitrogen mustard (notably in a pharmaceutical composition differing from the presently disclosed pharmaceutical composition).

The term “subject” refers to any vertebrate living organism. Especially, the subject is a mammal such as rodents including mice, rats, hamsters and guinea pigs; cats; dogs; rabbits; primates including marmosets; and humans. Most preferably the term “subject” refers to humans.

The term “essentially stable”, when referring to a pharmaceutical composition of bis-(2-chloroethyl)methylamine, means that at least about 90% of bis-(2-chloroethyl)methylamine is present in the composition (in other words less than about 10% of bis-(2-chloroethyl)methylamine has degraded) after storage at the specifically given conditions.

The term “partially exposed”, when referring to the exposure of a pharmaceutical composition to humidity and/or light, means that only a part of a given amount of a pharmaceutical composition is exposed to humidity and/or light. An illustrative, non-limiting example is the opening of an unsealed screw-capped tube containing the pharmaceutical composition in presence of humidity and/or light by removing the screw-cap.

It is understood that an excipient that exists in two different enantiomeric forms may be present in the pharmaceutical composition in one or the other enantiomerically pure form or in any mixture of both. Especially, such excipient may be present in racemic form. Lactic acid and menthol, if present, are preferably present in racemic form.

### EXAMPLES

### Example 1

### Pharmaceutical Composition Comprising bis-(2-chloroethyl)methylamine (Composition A)

| | Percent by weight |
|---|---|
| Component | of the composition |
| bis-(2-Chloroethyl)methylamine hydrochloride | 0.02% |
| Hydroxypropyl cellulose | 2% |
| Edetate disodium (dihydrate) | 0.01% |
| (DL) Menthol | 0.05% |
| Butylated hydroxytoluene | 0.01% |
| 2-(2-ethoxy-ethoxy)-ethanol | 49.91% |
| Isopropyl alcohol | 15.27% |
| Propylene glycol | 17.57% |
| Glycerin | 11.33% |
| Lactic acid (racemic) | 3.65% |
| Sodium chloride | 0.18% |
| Total | 100% |

### Example 2: Stability of Composition A Under Simulated In-Use Conditions

### (i) Analytical Methods

The assay of bis-(2-chloroethyl)methylamine in Composition A is accomplished with HPLC separation on a microbore column, followed by mass spectrometric detection in the select ion monitoring mode (peak area at Extracted Ion Chromatograms of 156) through comparison to an external standard calibration curve.

### (a) Used Instrumentation:

HPLC pump producing a flow rate of 0.2 mL/min
HPLC autosampler with 5° C. temperature control capability, 10 μL injection volume
HPLC column compartment with 25° C. temperature control capability
HPLC column: Waters Symmetry C18, 3.5 μm, 150×2.1 mm
Mass spectrometer: Agilent MSD using the following conditions:

| Ionization mode | Atmospheric pressure | |
|---|---|---|
| | ionization-electrospray (API-ES) | |
| Polarity | Positive | |
| VCap | 2000 | V |
| Detection Mode | Selective Ion Mode at | |
| | mass/charge ratio (m/z) 156 | |
| Fragmentor | 100 | |
| Gas temperature | 350° | C. |
| Drying gas | 12.0 | L/min |
| Nebulizer | 35 | psi |
| pressure | | |

### (b) Mobile Phase (MP):

| | |
|---|---|
| MP-A | water/acetonitrile/formic acid: 99/1/0.1 |
| MP-B | water/acetonitrile/formic acid: 1/99/0.1 |

gradient:

| time [min] | MP-A [%] | MP-B [%] |
|---|---|---|
| 0 | 100 | 0 |
| 3 | 100 | 0 |
| 20 | 0 | 100 |
| 23 | 0 | 100 |
| 23.1 | 100 | 0 |
| 30.0 | 100 | 0 |

### (ii) Experimental Details

The stability of Composition A under in-use conditions was assessed using different batches of Composition A filled in aluminum foil tubes containing 60 g each. All batches were stored in closed aluminum foil tubes (closed with a film seal and a cap closure) for different times at −25 to −15° C. before start of the in-use study: Batch A1 was stored for 7 month, Batch A2 for 36 month (differs from Batch A1 only in prolonged storage before start of in-use study), Batch B for 5 month and Batch C for 20 month.

At start of the in-use study the film seal of the tube was punctured, drug product content was dispensed from the tube, and the tube was resealed with the tube cap closure. The tubes were then exposed to conditions of 25° C./60% relative humidity for 1 hour (under opening of the tube, dispensing of a small amount of drug product and closing of the tube with the tube cap closure) and returned to refrigeration (2-8° C.) during each day of the in-use study. At several points in time (see table below) a sample of the drug product (Composition A) was taken and the assay of bis-(2-chloroethyl)methylamine in Composition A was analyzed according to the analytical methods above.

### (iii) Results

| | | Assay | | | |
|---|---|---|---|---|---|
| Timepoint | | Batch A1 | Batch A2 | Batch B | Batch C |
| Initial | | 102.0% | 98.3% | 97.4% | 100.2% |
| 1 | week | — | 95.0% | — | — |
| 2 | weeks | 102.6% | — | 100.8% | 99.1% |
| 4 | weeks | 102.4% | — | 101.7% | 101.6% |
| 8 | weeks | 96.9% | 97.4% | 100.9% | 94.9% |
| 12 | weeks | 97.9% | 99.2% | 98.1% | 94.5% |
| 16 | weeks | 98.8% | 95.8% | 93.8% | 100.0% |
| 20 | weeks | — | 92.8% | — | — |
| 24 | weeks | — | 92.9% | — | — |

1-20. (canceled)

21. A method of treating a skin disorder selected from cutaneous T-cell lymphoma, psoriasis, alopecia, lymphomatoid papulosis, parapsoriasis, Langerhans cell histiocytosis and vitiligo for a period of 90 days, the method comprising storing a pharmaceutical composition at or below 8° C.; topically applying the pharmaceutical composition to a subject in need thereof for a period of 90 days and storing the composition for up to 1 hour per day at about room temperature; and returning the composition for the remaining time of the day at or below 8° C., wherein the pharmaceutical composition comprises an effective amount of bis-(2-chloroethyl)methylamine, or a pharmaceutically acceptable salt thereof, and an excipient that is a compound of the formula HOCH₂CH₂OCH₂CH₂OR, wherein R represents (C₁-C₄)alkyl; and wherein at least 90% of the original amount of the bis-(2-chloroethyl)methylamine, or of the pharmaceutically acceptable salt thereof, is still present in the pharmaceutical composition, if the pharmaceutical composition is stored at about room temperature for up to 1 hour per day and at 8° C. or below for the remaining time of the day for a total of 90 days, and wherein the pharmaceutical composition is stored in a multiple-dose vial, container or tube;
wherein the bis-(2-chloroethyl)methylamine, or the pharmaceutically acceptable salt thereof, is initially present as bis-(2-chloroethyl)methylamine hydrochloride in an amount of about 0.01% to about 0.05% by weight of the composition.

22. The method according to claim 21, wherein the skin disorder is mycosis fungoides and wherein the bis-(2-chloroethyl)methylamine, or the pharmaceutically acceptable salt thereof, is present as bis-(2-chloroethyl)methylamine hydrochloride in an amount of about 0.01% to about 0.05% by weight of the composition.

23. The method according to claim 22, wherein the compound of the formula HOCH₂CH₂OCH₂CH₂OR is the compound HOCH₂CH₂OCH₂CH₂OCH₂CH₃; and wherein the compound HOCH₂CH₂OCH₂CH₂OCH₂CH₃ is present in an amount of about 40% to about 60% by weight of the composition.

24. The method according to claim 21, wherein at least 90% of the original amount of the bis-(2-chloroethyl)methylamine, or of the pharmaceutically acceptable salt thereof, is still present in the pharmaceutical composition, if the pharmaceutical composition is stored at about room temperature for up to 1 hour per day and at between 2° and 8° C. for the remaining time of the day for a total of 90 days.

25. The method according to claim 22, wherein at least 90% of the original amount of the bis-(2-chloroethyl)methylamine hydrochloride is still present in the pharmaceutical composition, if the pharmaceutical composition is stored at about room temperature for up to 1 hour per day and at between 2° and 8° C. for the remaining time of the day for a total of 90 days.

26. The method according to claim 23, wherein at least 90% of the original amount of the bis-(2-chloroethyl)methylamine hydrochloride is still present in the pharmaceutical composition, if the pharmaceutical composition is stored at about room temperature for up to 1 hour per day and at between 2° and 8° C. for the remaining time of the day for a total of 90 days.

27-30. (canceled)

31. The method according to claim 26, wherein the pharmaceutical composition further comprises hydroxypropyl cellulose, butylated hydroxytoluene, isopropyl alcohol and lactic acid.

32-34. (canceled)

35. The method according to claim 23, wherein the pharmaceutical composition further comprises:
about 1% to about 3% by weight of the composition hydroxypropyl cellulose;
about 0.005% to about 0.02% by weight of the composition edetate disodium dihydrate;
about 0.01% to about 0.1% by weight of the composition menthol;
about 0.005% to about 0.02% by weight of the composition butylated hydroxytoluene;
about 10% to about 20% by weight of the composition isopropyl alcohol;
about 13% to about 23% by weight of the composition propylene glycol;
about 6% to about 16% by weight of the composition glycerin;
about 2% to about 6% by weight of the composition lactic acid; and
about 0.1% to about 0.3% by weight of the composition sodium chloride.

36. The method according to claim 26, wherein the pharmaceutical composition further comprises:
about 1% to about 3% by weight of the composition hydroxypropyl cellulose;
about 0.005% to about 0.02% by weight of the composition edetate disodium dihydrate;
about 0.01% to about 0.1% by weight of the composition menthol;
about 0.005% to about 0.02% by weight of the composition butylated hydroxytoluene;
about 10% to about 20% by weight of the composition isopropyl alcohol;
about 13% to about 23% by weight of the composition propylene glycol;
about 6% to about 16% by weight of the composition glycerin;
about 2% to about 6% by weight of the composition lactic acid; and
about 0.1% to about 0.3% by weight of the composition sodium chloride.

37-40. (canceled)

41. The method according to claim 26, wherein the pharmaceutical composition is partially exposed to humidity and light during the period the pharmaceutical composition is stored at about room temperature.

42. (canceled)

43. The method according to claim 36, wherein the pharmaceutical composition is partially exposed to humidity and light during the period the pharmaceutical composition is stored at about room temperature.

44-48. (canceled)

49. The method according to claim 21, wherein the skin disorder is Stage IA and IB mycosis fungoides-type cutaneous T-cell lymphoma.

50. The method according to claim 49, wherein the subject has received prior skin-directed therapy.

51. The method according to claim 21, wherein the pharmaceutical composition is stored in a multiple-dose vial, container or tube and the vial, container or tube can be used to dispense treatment for 90 to 240 days.

52. The method of claim 21, wherein the bis-(2-chloroethyl)methylamine, or the pharmaceutically acceptable salt thereof, is initially present as bis-(2-chloroethyl)methylamine hydrochloride in an amount of about 0.02% by weight of the composition.

53. The method of claim 22, wherein the bis-(2-chloroethyl)methylamine, or the pharmaceutically acceptable salt thereof, is initially present as bis-(2-chloroethyl)methylamine hydrochloride in an amount of about 0.02% by weight of the composition.

54. The method of claim 23, wherein the bis-(2-chloroethyl)methylamine, or the pharmaceutically acceptable salt thereof, is initially present as bis-(2-chloroethyl)methylamine hydrochloride in an amount of about 0.02% by weight of the composition.

55. The method of claim 26, wherein the bis-(2-chloroethyl)methylamine, or the pharmaceutically acceptable salt thereof, is initially present as bis-(2-chloroethyl)methylamine hydrochloride in an amount of about 0.02% by weight of the composition.

56. The method of claim 31, wherein the bis-(2-chloroethyl)methylamine, or the pharmaceutically acceptable salt thereof, is initially present as bis-(2-chloroethyl)methylamine hydrochloride in an amount of about 0.02% by weight of the composition.

57. The method of claim 49, wherein the bis-(2-chloroethyl)methylamine, or the pharmaceutically acceptable salt thereof, is initially present as bis-(2-chloroethyl)methylamine hydrochloride in an amount of about 0.02% by weight of the composition.

61. The method of claim 51, wherein the bis-(2-chloroethyl)methylamine, or the pharmaceutically acceptable salt thereof, is initially present as bis-(2-chloroethyl)methylamine hydrochloride in an amount of about 0.02% by weight of the composition.
